(19) United States
(12) Patent Application Publication
(10) Pub. No.: US 2007/0104792 A1
(43) Pub. Date: May 10, 2007

(54) Nanoparticulate tadalafil formulations

(75) Inventor: Scott Jenkins, Downingtown, PA (US)

Correspondence Address: ELAN DRUG DELIVERY, INC.;C/O FOLEY & LARDNER LLP, 3000 K STREET, N.W., SUITE 500, WASHINGTON, DC, 20007-5109, US

(73) Assignee: Elan Pharma International, Limited

(21) Appl. No.: 11/520,059

(22) Filed: Sep. 13, 2006

Related U.S. Application Data
(60) Provisional application No. 60/716,405, filed on Sep. 13, 2005.

(51) Int. Cl. A61K 9/14

(52) U.S. Cl. 424/489

(57) ABSTRACT

The present invention is directed to compositions comprising nanoparticulate tadalafil, or a salt or derivative thereof, having improved bioavailability, faster rates of absorption and a faster onset of therapeutic effect. The nanoparticulate tadalafil particles of the composition are proposed to have an effective average particle size of less than about 2000 nm and may be useful in the treatment of sexual dysfunction and vascular-, pulmonary- and cardiac-related diseases and conditions.

### CROSS-REFERENCE TO RELATED PATENT APPLICATIONS

This application claims the benefit under 35 U.S.C. § 119(e) to U.S. Provisional Application No. 60/716,405, filed on Sep. 13, 2005 which is incorporated by reference herein in its entirety.

### FIELD OF INVENTION

The present invention relates generally to compounds and compositions useful in the treatment of sexual dysfunction and other cardiovascular-, pulmonary- or vascular-related conditions. More specifically, the invention relates to nanoparticulate PDE5 inhibitor compositions, such as nanoparticulate tadalafil, or salts or derivatives thereof, having an effective average particle size of less than about 2000 nm. The invention also relates to nanoparticulate PDE5 inhibitor formulations, methods of manufacturing nanoparticulate PDE5 inhibitor compositions, and methods of treatment using such compositions.

### BACKGROUND

A. Background Regarding Tadalafil

Tadalafil, one of a class of cyclic guanosine monophosphate (“cGMP”) specific phosphodiesterase type 5 (“PDE5”) inhibitors, is most know for its use as a systemic impotence therapy agent, generally used to treat erectile dysfunction in men by increasing blood flow. The prescription PDE5 inhibitors (e.g., sildenafil (Viagra®), vardenafil (Levitra®) and tadalafil (Cialis®)) act by blocking the ability of PDE5 to degrade cGMP. cGMP permits the smooth muscle inside the arteries in the penis to relax, thus allowing blood flow to the corpus cavernosum to increase.

The increased blood flow provided by PDE5 5 inhibitors has also been used to treat sexual dysfunction in women, and has been used in the treatment of other medical conditions or diseases, such as pulmonary arterial hypertension (e.g., in conjunction with a prostacyclin) and/or the effects and symptoms of myocardial infarction. For example, PDE5 5 inhibitors may be used for the prevention of ischemia/reperfusion injury, for example, in patients undergoing heart surgery. Administration of PDE5 5 inhibitors such as tadalafil can also be administered to subjects during or after a heart attack (myocardial infarction) to prevent or lessen ischemic heart damage.

Another use for PDE5 5 inhibitors involves improving pulmonary perfusion. In patients with inflammatory and degenerative lung disorders such as, for example, chronic obstructive pulmonary disease (CQPD), adult respiratory distress syndrome (ARDS), acute lung injury (ALI), bronchitis, bronchial asthma, pulmonary fibroses, emphysema, interstitial pulmonary disorders and pneumonias there is observed to be partial or global respiratory failure. PDE5 5 inhibitors, such as tadalafil, may be administered to patients suffering from such conditions or disease, to alleviate or reduce patient symptoms.

Tadalafil is chemically known as pyrazino[1′,2′:1,6]pyrido[3,4-b]indole-1,4-dione, 6-(1,3-benzodioxol-5-yl)-2,3,6,7,12,12a-hexahydro-2-methyl-, (6R,12aR) with an empirical formula of C₂₂H₁₉N₃O₄. Tadalafil has a molecular weight of 389.41, and the chemical structure shown below:

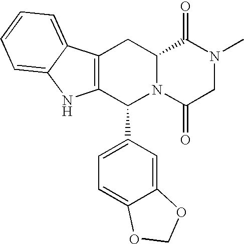

The conventional formulation of tadalafil is a crystalline solid that is practically insoluble in water and very slightly soluble in ethanol. Tadalafil is commercially available from Lilly ICOS under the brand name Cialis®. Cialis® is manufactured for Lilly ICOS LLC by Eli Lilly and Company of Indianapolis, Ind. Cialis® is available as film-coated, almond-shaped tablets for oral administration in strengths of 5 mg, 10 mg or 20 mg of tadalafil. Cialis® tablets contain inactive ingredients of croscarmellose sodium, hydroxypropyl cellulose, hypromellose, iron oxide, lactose monohydrate, magnesium stearate, microcrystalline cellulose, sodium lauryl sulfate, talc, titanium dioxide, and triacetin.

Dosing of tadalafil varies by patient; however, it is generally administered in 10 mg dosages taken within 36 hours prior to sexual activity. The dose may be increased to 20 mg or decreased to 5 mg, based on individual efficacy and tolerance. The maximum recommended dosing frequency is once per day in most patients. Cialis® may be taken without regard to food.

PDE5 inhibitors such as tadalafil are not recommended for subjects taking any medication that contains nitrates, such as nitroglycerin; the combination may result in a dangerous lowering of blood pressure, possibly causing stroke, a heart attack, or death. Additionally, alpha-blockers, used to treat high blood pressure or an enlarged prostate, may also contraindicate PDE5 inhibitors.

Other side effects—rare and usually temporary—may include headache, skin flushing, indigestion, nasal congestion and affected vision (e.g, bluish tinge to vision or light sensitivity).

Tadalafil compounds have been disclosed in, for example, U.S. Pat. No. 5,859,006 to Daugan for “Tetracyclic Derivatives; Process of Preparation and Use,” U.S. Pat. No. 6,140,329 to Daugan for “Use of cGMP-Phosphodiesterase Inhibitors in Methods and Compositions to Treat Impotence,” U.S. Pat. No. 6,821,975 to Anderson et al. for “Beta-Carboline Drug Products,” U.S. Pat. Nos. 6,809,112; 6,890,945; 6,903,127 and 6,921,771 to McCall et al. for “Method of Treating Sexual Disturbances,” U.S. Pat. No. 6,803,031 to Rabinowitz et al. for “Delivery of Erectile Dysfunction Drugs Through an Inhalation Route,” U.S. Pat. No. 6,548,490 to Doherty, Jr., et al. for “Transmucosal Administration of Phosphodiesterase Inhibitors for the Treatment of Erectile Dysfunction,” U.S. Pat. No. 6,469,016 to Place et al. for “Treatment of Female Sexual Dysfunction Using Phosphodiesterase Inhibitors,” U.S. Pat. No. 7,091,207 to Kukreja for “Methods of Treating Myocardial Infarction with PDE-5 Inhibitors,” U.S. Patent Pub. No. 20060148693 to Wollin for “Composition Comprising a Pulmonary Surfactant and a PDE-5 Inhibitor for the Treatment of Lung Diseases,” and U.S. Patent Pub. No. 20050101608 to Donald for “Iloprost in Combination Therapies for the Treatment of Pulmonary Arterial Hypertension.”

Because tadalafil is practically insoluble in water, the dissolution rate and bioavailability of conventional tadalafil (such as Cialis) formulations are likely poor. Thus, it would be desirable to increase the dissolution rate and bioavailability for faster drug onset. The present invention fulfills such needs by providing nanoparticulate tadalafil compositions which overcome these and other shortcomings of conventional formulations.

The present invention then, relates to nanoparticulate PDE5 inhibitor, such as tadalafil or salts or derivatives thereof, compositions for the treatment of sexual dysfunction, such as erectile dysfunction, and other cardiac-, pulmonary- and vascular-related conditions.

B. Background Regarding Nanoparticulate Active Agent Compositions

Nanoparticulate active agent compositions, first described in U.S. Pat. No. 5,145,684 (“the '684 patent”), comprise particles of a poorly soluble therapeutic or diagnostic agent having adsorbed onto or associated with the surface thereof a non-crosslinked surface stabilizer. The '684 patent also describes method of making such nanoparticulate active agent compositions but does not describe compositions comprising tadalafil in nanoparticulate form. Methods of making nanoparticulate active agent compositions are described in, for example, U.S. Pat. Nos. 5,518,187 and 5,862,999, both for “Method of Grinding Pharmaceutical Substances”; U.S. Pat. No. 5,718,388, for “Continuous Method of Grinding Pharmaceutical Substances”; and U.S. Pat. No. 5,510,118 for “Process of Preparing Therapeutic Compositions Containing Nanoparticles.”

Nanoparticulate active agent compositions are also described, for example, in U.S. Pat. No. 5,298,262 for “Use of Ionic Cloud Point Modifiers to Prevent Particle Aggregation During Sterilization”; U.S. Pat. No. 5,302,401 for “Method to Reduce Particle Size Growth During Lyophilization”; U.S. Pat. No. 5,318,767 for “X-Ray Contrast Compositions Useful in Medical Imaging”; U.S. Pat. No. 5,326,552 for “Novel Formulation For Nanoparticulate X-Ray Blood Pool Contrast Agents Using High Molecular Weight Non-ionic Surfactants”; U.S. Pat. No. 5,328,404 for “Method of X-Ray Imaging Using lodinated Aromatic Propanedioates”; U.S. Pat. No. 5,336,507 for “Use of Charged Phospholipids to Reduce Nanoparticle Aggregation”; U.S. Pat. No. 5,340,564 for “Formulations Comprising Olin 10-G to Prevent Particle Aggregation and Increase Stability”; U.S. Pat. No. 5,346,702 for “Use of Non-Ionic Cloud Point Modifiers to Minimize Nanoparticulate Aggregation During Sterilization”; U.S. Pat. No. 5,349,957 for “Preparation and Magnetic Properties of Very Small Magnetic-Dextran Particles”; U.S. Pat. Nos. 5,352,459 for “Use of Purified Surface Modifiers to Prevent Particle Aggregation During Sterilization”; U.S. Pat. No. 5,399,363 and 5,494,683, both for “Surface Modified Anticancer Nanoparticles”; U.S. Pat. No. 5,401,492 for “Water Insoluble Non-Magnetic Manganese Particles as Magnetic Resonance Enhancement Agents”; U.S. Pat. No. 5,429,824 for “Use of Tyloxapol as a Nanoparticulate Stabilizer”; U.S. Pat. No. 5,447,710 for “Method for Making Nanoparticulate X-Ray Blood Pool Contrast Agents Using High Molecular Weight Non-ionic Surfactants”; U.S. Pat. No. 5,451,393 for “X-Ray Contrast Compositions Useful in Medical Imaging”; U.S. Pat. No. 5,466,440 for “Formulations of Oral Gastrointestinal Diagnostic X-Ray Contrast Agents in Combination with Pharmaceutically Acceptable Clays”; U.S. Pat. No. 5,470,583 for “Method of Preparing Nanoparticle Compositions Containing Charged Phospholipids to Reduce Aggregation”; U.S. Pat. No. 5,472,683 for “Nanoparticulate Diagnostic Mixed Carbamic Anhydrides as X-Ray Contrast Agents for Blood Pool and Lymphatic System Imaging”; U.S. Pat. No. 5,500,204 for “Nanoparticulate Diagnostic Dimers as X-Ray Contrast Agents for Blood Pool and Lymphatic System Imaging”; U.S. Pat. No. 5,518,738 for “Nanoparticulate NSAID Formulations”; U.S. Pat. No. 5,521,218 for “Nanoparticulate Iododipamide Derivatives for Use as X-Ray Contrast Agents”; U.S. Pat. No. 5,525,328 for “Nanoparticulate Diagnostic Diatrizoxy Ester X-Ray Contrast Agents for Blood Pool and Lymphatic System Imaging”; U.S. Pat. No. 5,543,133 for “Process of Preparing X-Ray Contrast Compositions Containing Nanoparticles”; U.S. Pat. No. 5,552,160 for “Surface Modified NSAID Nanoparticles”; U.S. Pat. No. 5,560,931 for “Formulations of Compounds as Nanoparticulate Dispersions in Digestible Oils or Fatty Acids”; U.S. Pat. No. 5,565,188 for “Polyalkylene Block Copolymers as Surface Modifiers for Nanoparticles”; U.S. Pat. No. 5,569,448 for “Sulfated Non-ionic Block Copolymer Surfactant as Stabilizer Coatings for Nanoparticle Compositions”; U.S. Pat. No. 5,571,536 for “Formulations of Compounds as Nanoparticulate Dispersions in Digestible Oils or Fatty Acids”; U.S. Pat. No. 5,573,749 for “Nanoparticulate Diagnostic Mixed Carboxylic Anydrides as X-Ray Contrast Agents for Blood Pool and Lymphatic System Imaging”; U.S. Pat. No. 5,573,750 for “Diagnostic Imaging X-Ray Contrast Agents”; U.S. Pat. No. 5,573,783 for “Redispersible Nanoparticulate Film Matrices With Protective Overcoats”; U.S. Pat. No. 5,580,579 for “Site-specific Adhesion Within the GI Tract Using Nanoparticles Stabilized by High Molecular Weight, Linear Poly(ethylene Oxide) Polymers”; U.S. Pat. No. 5,585,108 for “Formulations of Oral Gastrointestinal Therapeutic Agents in Combination with Pharmaceutically Acceptable Clays”; U.S. Pat. No. 5,587,143 for “Butylene Oxide-Ethylene Oxide Block Copolymers Surfactants as Stabilizer Coatings for Nanoparticulate Compositions”; U.S. Pat. No. 5,591,456 for “Milled Naproxen with Hydroxypropyl Cellulose as Dispersion Stabilizer”; U.S. Pat. No. 5,593,657 for “Novel Barium Salt Formulations Stabilized by Non-ionic and Anionic Stabilizers”; U.S. Pat. No. 5,622,938 for “Sugar Based Surfactant for Nanocrystals”; U.S. Pat. No. 5,628,981 for “Improved Formulations of Oral Gastrointestinal Diagnostic X-Ray Contrast Agents and Oral Gastrointestinal Therapeutic Agents”; U.S. Pat. No. 5,643,552 for “Nanoparticulate Diagnostic Mixed Carbonic Anhydrides as X-Ray Contrast Agents for Blood Pool and Lymphatic System Imaging”; U.S. Pat. No. 5,718,388 for “Continuous Method of Grinding Pharmaceutical Substances”; U.S. Pat. No. 5,718,919 for “Nanoparticles Containing the R(−)Enantiomer of Ibuprofen”; U.S. Pat. No. 5,747,001 for “Aerosols Containing Beclomethasone Nanoparticle Dispersions”; U.S. Pat. No. 5,834,025 for “Reduction of Intravenously Administered Nanoparticulate Formulation Induced Adverse Physiological Reactions”; U.S. Pat. No. 6,045,829 “Nanocrystalline Formulations of Human Immunodeficiency Virus (HIV) Protease Inhibitors Using Cellulosic Surface Stabilizers”; U.S. Pat. No. 6,068,858 for “Methods of Making Nanocrystalline Formulations of Human Immunodeficiency Virus (HIV) Protease Inhibitors Using Cellulosic Surface Stabilizers”; U.S. Pat. No. 6,153,225 for “Injectable Formulations of Nanoparticulate Naproxen”; U.S. Pat. No. 6,165,506 for “New Solid Dose Form of Nanoparticulate Naproxen”; U.S. Pat. No. 6,221,400 for “Methods of Treating Mammals Using Nanocrystalline Formulations of Human Immunodeficiency Virus (HIV) Protease Inhibitors”; U.S. Pat. No. 6,264,922 for “Nebulized Aerosols Containing Nanoparticle Dispersions”; U.S. Pat. No. 6,267,989 for “Methods for Preventing Crystal Growth and Particle Aggregation in Nanoparticle Compositions”; U.S. Pat. No. 6,270,806 for “Use of PEG-Derivatized Lipids as Surface Stabilizers for Nanoparticulate Compositions”; U.S. Pat. No. 6,316,029 for “Rapidly Disintegrating Solid Oral Dosage Form,” U.S. Pat. No. 6,375,986 for “Solid Dose Nanoparticulate Compositions Comprising a Synergistic Combination of a Polymeric Surface Stabilizer and Dioctyl Sodium Sulfosuccinate”; U.S. Pat. No. 6,428,814 for “Bioadhesive Nanoparticulate Compositions Having Cationic Surface Stabilizers”; U.S. Pat. No. 6,431,478 for “Small Scale Mill”; U.S. Pat. No. 6,432,381 for “Methods for Targeting Drug Delivery to the Upper and/or Lower Gastrointestinal Tract,” U.S. Pat. No. 6,582,285 for “Apparatus for Sanitary Wet Milling”; and U.S. Pat. No. 6,592,903 for “Nanoparticulate Dispersions Comprising a Synergistic Combination of a Polymeric Surface Stabilizer and Dioctyl Sodium Sulfosuccinate”; U.S. Pat. No. 6,656,504 for “Nanoparticulate Compositions Comprising Amorphous Cyclosporine”; U.S. Pat. No. 6,742,734 for “System and Method for Milling Materials”; U.S. Pat. No. 6,745,962 for “Small Scale Mill and Method Thereof”; U.S. Pat. No. 6,811,767 for “Liquid Droplet Aerosols of Nanoparticulate Drugs”; U.S. Pat. No. 6,908,626 for “Compositions Having a Combination of Immediate Release and Controlled Release Characteristics”; U.S. Pat. No. 6,969,529 for “Nanoparticulate Compositions Comprising Copolymers of Vinyl Pyrrolidone and Vinyl Acetate as Surface Stabilizers”; U.S. Pat. No. 6,976,647 for “System and Method for Milling Materials”; and U.S. Pat. No. 6,991,191 for “Method of Using a Small Scale Mill”; all of which are specifically incorporated by reference.

In addition, U.S. Patent Publication No. 20020012675 A1, for “Controlled Release Nanoparticulate Compositions”; U.S. Patent Publication No. 20050276974 for “Nanoparticulate Fibrate Formulations”; U.S. Patent Publication No. 20050238725 for “Nanoparticulate Compositions Having a Peptide as a Surface Stabilizer”; U.S. Patent Publication No. 20050233001 for “Nanoparticulate Megestrol Formulations”; U.S. Patent Publication No. 20050147664 for “Compositions Comprising Antibodies and Methods of Using the Same for Targeting Nanoparticulate Active Agent Delivery”; U.S. Patent Publication No. 20050063913 for “Novel Metaxalone Compositions”; U.S. Patent Publication No. 20050042177 for “Novel Compositions of Sildenafil Free Base”; U.S. Patent Publication No. 20050031691 for “Gel Stabilized Nanoparticulate Active Agent Compositions”; U.S. Patent Publication No. 20050019412 for “Novel Glipizide Compositions”; U.S. Patent Publication No. 20050004049 for “Novel Griseofulvin Compositions”; U.S. Patent Publication No. 20040258758 for “Nanoparticulate Topiramate Formulations”; U.S. Patent Publication No. 20040258757 for “Liquid Dosage Compositions of Stable Nanoparticulate Active Agents”; U.S. Patent Publication No. 20040229038 for “Nanoparticulate Meloxicam Formulations”; U.S. Patent Publication No. 20040208833 for “Novel Fluticasone Formulations”; U.S. Patent Publication No. 20040195413 for “Compositions and Method for Milling Materials”; U.S. Patent Publication No. 20040156895 for “Solid Dosage Forms Comprising Pullulan”; U.S. Patent Publication No. 20040156872 for “Novel Nimesulide Compositions”; U.S. Patent Publication No. 20040141925 for “Novel Triamcinolone Compositions”; U.S. Patent Publication No. 20040115134 for “Novel Nifedipine Compositions”; U.S. Patent Publication No. 20040105889 for “Low Viscosity Liquid Dosage Forms”; U.S. Patent Publication No. 20040105778 for “Gamma Irradiation of Solid Nanoparticulate Active Agents”; U.S. Patent Publication No. 20040101566 for “Novel Benzoyl Peroxide Compositions”; U.S. Patent Publication No. 20040057905 for “Nanoparticulate Beclomethasone Dipropionate Compositions”; U.S. Patent Publication No. 20040033267 for “Nanoparticulate Compositions of Angiogenesis Inhibitors”; U.S. Patent Publication No. 20040033202 for “Nanoparticulate Sterol Formulations and Novel Sterol Combinations”; U.S. Patent Publication No. 20040018242 for “Nanoparticulate Nystatin Formulations”; U.S. Patent Publication No. 20040015134 for “Drug Delivery Systems and Methods”; U.S. Patent Publication No. 20030232796 for “Nanoparticulate Polycosanol Formulations & Novel Polycosanol Combinations”; U.S. Patent Publication No. 20030215502 for “Fast Dissolving Dosage Forms Having Reduced Friability”; U.S. Patent Publication No. 20030185869 for “Nanoparticulate Compositions Having Lysozyme as a Surface Stabilizer”; U.S. Patent Publication No. 20030181411 for “Nanoparticulate Compositions of Mitogen-Activated Protein (MAP) Kinase Inhibitors”; U.S. Patent Publication No. 20030137067 for “Compositions Having a Combination of Immediate Release and Controlled Release Characteristics”; U.S. Patent Publication No. 20030108616 for “Nanoparticulate Compositions Comprising Copolymers of Vinyl Pyrrolidone and Vinyl Acetate as Surface Stabilizers”; U.S. Patent Publication No. 20030095928 for “Nanoparticulate Insulin”; U.S. Patent Publication No. 20030087308 for “Method for High Through- put Screening Using a Small Scale Mill or Microfluidics”; U.S. Patent Publication No. 20030023203 for “Drug Delivery Systems & Methods”; U.S. Patent Publication No. 20020179758 for “System and Method for Milling Materials”; and U.S. Patent Publication No. 20010053664 for “Apparatus for Sanitary Wet Milling,” describe nanoparticulate active agent compositions and are specifically incorporated by reference. None of these references describe compositions of nanoparticulate PDE5 inhibitors such as tadalafil.

Amorphous small particle compositions are described, for example, in U.S. Pat. No. 4,783,484 for “Particulate Composition and Use Thereof as Antimicrobial Agent”; U.S. Pat. No. 4,826,689 for “Method for Making Uniformly Sized Particles from Water-Insoluble Organic Compounds”; U.S. Pat. No. 4,997,454 for “Method for Making Uniformly-Sized Particles From Insoluble Compounds”; U.S. Pat. No. 5,741,522 for “Ultrasmall, Non-aggregated Porous Particles of Uniform Size for Entrapping Gas Bubbles Within and Methods”; and U.S. Pat. No. 5,776,496, for “Ultrasmall Porous Particles for Enhancing Ultrasound Back Scatter,” all of which are specifically incorporated herein by reference.

Tadalafil has high therapeutic value in the treatment of sexual dysfunctions, such as erectile dysfunction. It is also useful for the treatment of cardiac, pulmonary and vascular-related conditions. However, because it is practically insoluble in water, the dissolution of conventional microcrystalline tadalafil tablets is poor in aqueous (e.g., physiological) environments. Thus, tadalafil has limited bioavailability, which limits the therapeutic outcome for treatments requiring tadalafil. Accordingly, there is a need in the art for tadalafil formulations which overcome this and other problems associated with its use. A tadalafil composition which exhibits enhanced bioavailability, increased dissolution rate, reduced drug dosage, and reduced adverse side effects would satisfy these needs.

### SUMMARY

The compositions and methods described herein relate to compositions comprising at least one nanoparticulate PDE5 inhibitor, such as tadalafil, or a salt or derivative thereof (referred to herein collectively as tadalafil), having an effective average particle size of less than about 2000 nm. In general, the compositions comprise particles of a nanoparticulate PDE5 inhibitor, and at least one surface stabilizer adsorbed or associated with the surface of the PDE5 inhibitor particles. Such nanoparticles may be in crystalline phase, an amorphous phase, a semi-crystalline phase, a semi-amorphous phase, and mixtures thereof.

Additionally, the compositions may comprise one or more surface stabilizers. For example, the compositions may comprise at least one primary and at least one secondary surface stabilizer. Exemplary surface stabilizers may include one or more of an anionic surface stabilizer, a cationic surface stabilizers, a non-ionic surface stabilizers, a zwitterionic surface stabilizers, and an ionic surface stabilizers.

In some embodiments, the compositions may additionally include one or more pharmaceutically acceptable excipients, carriers, active agents or combinations thereof. In some embodiments, active agents may include agents useful for the treatment of sexual dysfunction and cardiac-, pulmonary- and vascular-related conditions. By way of example but not by way of limitation, active agents may include sildenafil, vardenafil, testosterone, bremlanotide, ginseng and combinations thereof.

Additionally disclosed are methods related to making nanoparticulate PDE5 inhibitor (such as tadalafil) compositions having an effective average particle size of less than about 2000 nm. By way of example, but not by way of limitation, methods may include contacting particles of the tadalafil with at least one surface stabilizer for a time and under conditions sufficient to provide a nanoparticulate tadalafil composition having an effective average particle size of less than about 2000 nm. In some methods, contacting may include, for example, milling, homogenization, freezing, template emulsion, precipitation, supercritical fluid techniques or combinations thereof.

The nanoparticulate PDE5 inhibitor compositions described herein may be formulated for dosage or administration in a variety of forms, although in some embodiments, a solid dosage form may be preferred (e.g., to treat the symptoms of erectile dysfunction or other sexual dysfunction); a cream, gel, or bioadhesive form may be preferred (e.g., to treat the symptoms of sexual dysfunction in men or women, or to treat cardiac or pulmonary conditions); an aerosol or inhaled form may be preferred (e.g., for rapid pulmonary delivery); or an injectable form may be preferred (e.g., for rapid cardiac, vascular or pulmonary delivery). Though any pharmaceutically acceptable dosage form may be utilized, dosage forms contemplated include but are not limited to formulations for oral, pulmonary, rectal, colonic, parenteral, intracisternal, intravaginal, intraperitoneal, ocular, otic, local, buccal, nasal, and topical administration. Dosage forms may include bioadhesives, liquid dispersions, gels, aerosols, ointments, creams, lyophilized formulations, tablets, and capsules, and dosage forms may also include controlled release formulations, fast melt formulations, delayed release formulations, extended release formulations, pulsatile release formulations, and mixed immediate release and controlled release formulations. Combinations of these dosage forms are also contemplated.

The nanoparticulate PDE5 inhibitor compositions disclosed herein are also contemplated to exhibit improved pharmacokinetic properties as compared to a non-nanoparticulate composition of the same PDE5 inhibitor.

In further embodiments, the pharmacokinetic profiles of the nanoparticulate PDE5 inhibitor compositions may be substantially similar when administered to a fed or fasted subject; in other embodiments, the nanoparticulate PDE5 inhibitor compositions may be bioequivalent when administered to a fed or fasted subject.

Also disclosed are methods of using the nanoparticulate PDE5 inhibitor formulations, for example, to treat or prevent diseases, disorders, symptoms or conditions in a subject. By way of example, but not by way of limitation, the compositions may be used to treat sexual dysfunction in men and women, (e.g., erectile dysfunction in men), vascular disorders or diseases such as pulmonary arterial hypertension, the effects and symptoms of myocardial infarction, ischemia/reperfusion injury, inflammatory and degenerative lung disorders, for example, chronic obstructive pulmonary disease (COPD), adult respiratory distress syndrome (ARDS), acute lung injury (ALI), bronchitis, bronchial asthma, pulmonary fibroses, emphysema, interstitial pulmonary disorders and pneumonias.

Exemplary methods of treatment may include administering to a subject a stable nanoparticulate PDE5 inhibitor (such as tadalafil) composition including at least one PDE5 inhibitor or derivative or salt thereof and at least one surface stabilizer having an effective average particle size of less than about 200 nm. In some embodiments, the subject may have been diagnosed with a sexual dysfunction, such as erectile dysfunction, or a condition, disease or symptoms related to cardiac, pulmonary or vascular function. In other embodiments, the compositions may be used to treat symptoms indicative of sexual dysfunction, such as erectile dysfunction, and other vascular-, cardiac- and/or pulmonary-related condition.

Both the foregoing summary of the invention and the following detailed description of the invention are exemplary and explanatory and are intended to provide further details of the invention as claimed. Other objects, advantages, and novel features will be readily apparent to those skilled in the art from the following detailed description of the invention.

### DETAILED DESCRIPTION

A. Nanoparticulate Tadalafil Compositions

The compositions described herein include nanoparticulate PDE5 inhibitors such as tadalafil or a salt or derivative thereof, and preferably at least one surface stabilizer associated with or adsorbed on the surface of the drug. In some embodiments, the tadalafil particles may have an effective average particle size of less than about 2000 nm.

As taught by the '684 patent, and as described in more detail below, not every combination of surface stabilizer and active agent will result in a stable nanoparticulate composition. Thus, it was surprisingly discovered that stable, nanoparticulate tadalafil formulations can be made.

Advantages of the nanoparticulate tadalafil formulation of the invention as compared to non-nanoparticulate tadalafil compositions (e.g., microcrystalline or solubilized dosage forms) may include, but are not limited to, one or more of the following: (1) smaller tablet or other solid dosage form size; (2) smaller doses of drug required to obtain the same pharmacological effect; (3) improved pharmacokinetic profiles, (4) increased bioavailability; (5) substantially similar pharmacokinetic profiles of the nanoparticulate tadalafil compositions when administered in the fed versus the fasted state; (6) bioequivalency of the nanoparticulate tadalafil compositions when administered in the fed versus the fasted state; (7) an increased rate of dissolution for the tadalafil compositions; and (8) the use of nanoparticulate tadalafil compositions in conjunction with other active agents useful in the treatment of sexual dysfunction such as erectile dysfunction or cardiac-, pulmonary- or vascular-related conditions, diseases or disorders.

The present invention also relates to nanoparticulate tadalafil compositions together with one or more non-toxic physiologically acceptable carriers, adjuvants, or vehicles, collectively referred to as carriers.

The nanoparticulate PDE5 inhibitors, such as tadalafil may be formulated for administration in a variety of forms. For example, the compositions may be formulated for parental injection (e.g., intravenous, intramuscular, or subcutaneous), oral administration in solid, liquid, bioadhesive or aerosol form, vaginal, nasal, rectal, ocular, local (powders, ointments, or drops), buccal, intracistemal, intraperitoneal, or topical administrations, and the like.

Although any pharmaceutically acceptable dosage form can be utilized, in some embodiments, a preferred dosage form may be a solid dosage form such as a tablet. In other embodiments, preferred solid dosage forms may include, but are not limited to, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof.

The present invention is described herein using several definitions, as set forth below and throughout the application.

As used herein, the term “subject” is used to mean an animal, preferably a mammal, including a human or non-human. The terms patient and subject may be used interchangeably.

The term “effective average particle size of less than about 2000 nm,” as used herein, means that at least about 50% of the nanoparticulate tadalafil particles have a size of less than about 2000 nm (by weight or by other suitable measurement technique, such as by number or by volume) when measured by, for example, sedimentation flow fractionation, photon correlation spectroscopy, light scattering, disk centrifugation, and other techniques known to those of skill in the art.

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein with reference to stable nanoparticulate tadalafil, “stable” connotes, but is not limited to one or more of the following parameters: (1) the particles do not appreciably flocculate or agglomerate due to interparticle attractive forces or otherwise significantly increase in particle size over time; (2) that the physical structure of the particles is not altered over time, such as by conversion from an amorphous phase to a crystalline phase; (3) that the particles are chemically stable; and/or (4) where the tadalafil has not been subject to a heating step at or above the melting point of the tadalafil in the preparation of the nanoparticles of the present invention.

The term “conventional” or “non-nanoparticulate” active agent shall mean an active agent which is solubilized or which has an effective average particle size of greater than about 2000 nm. Nanoparticulate active agents as defined herein have an effective average particle size of less than about 2000 nm.

The phrase “poorly water soluble drugs” as used herein refers to those drugs that have a solubility in water of less than about 30 mg/ml, less than about 20 mg/ml, less than about 10 mg/ml, or less than about 1 mg/ml.

As used herein, the phrase “therapeutically effective amount” shall mean that drug dosage that provides the specific pharmacological response for which the drug is administered in a significant number of subjects in need of such treatment. It is emphasized that a therapeutically effective amount of a drug that is administered to a particular subject in a particular instance will not always be effective in treating the conditions/diseases described herein, even though such dosage is deemed to be a therapeutically effective amount by those of skill in the art.

The term “particulate” as used herein refers to a state of matter which is characterized by the presence of discrete particles, pellets, beads or granules irrespective of their size, shape or morphology. The term “multiparticulate” as used herein means a plurality of discrete or aggregated particles, pellets, beads, granules or mixtures thereof irrespective of their size, shape or morphology.

B. Preferred Characteristics of the Nanoparticulate Tadalafil Compositions

1. Increased Bioavailability

The compositions of nanoparticulate PDE5 inhibitors, such as tadalafil, or a salt or derivative thereof, are proposed to exhibit increased bioavailability, and require smaller doses as compared to prior or conventional tadalafil formulations.

In some embodiments, the nanoparticulate tadalafil compositions, upon administration to a mammal (e.g., a human, for example a human male diagnosed with erectile dysfunction), produce therapeutic results at a dosage which is less than that of a non-nanoparticulate dosage form of the same tadalafil. Additionally, because the dose sizes of nanoparticulate formulations are contemplated to be smaller than conventional dosages, adverse side-effects are expected to be reduced or eliminated with the nanoparticulate formulations.

2. Improved Pharmacokinetic Profiles

The nanoparticulate PDE5 inhibitor compositions, such as tadalafil, described herein may also exhibit a desirable pharmacokinetic profile when administered to mammalian subjects. The desirable pharmacokinetic profile of the tadalafil compositions preferably includes, but is not limited to: (1) a Cₘₐₓ for tadalafil or a derivative or salt thereof, when assayed in the plasma of a mammalian subject following administration, that is preferably greater than the Cₘₐₓ for a non-nanoparticulate formulation of the same tadalafil, administered at the same dosage; and/or (2) an AUC for tadalafil or a derivative or a salt thereof, when assayed in the plasma of a mammalian subject following administration, that is preferably greater than the AUC for a non-nanoparticulate formulation of the same tadalafil, administered at the same dosage; and/or (3) a Tₘₐₓ for tadalafil or a derivative or a salt thereof, when assayed in the plasma of a mammalian subject following administration, that is preferably less than the Tₘₐₓ for a non-nanoparticulate formulation of the same tadalafil, administered at the same dosage. The desirable pharmacokinetic profile, as used herein, is the pharmacokinetic profile measured after the initial dose of tadalafil or derivative or a salt thereof.

In one embodiment, a composition comprising at least one nanoparticulate tadalafil or a derivative or salt thereof exhibits in comparative pharmacokinetic testing with a non-nanoparticulate formulation of the same tadalafil (e.g., Cialis®), administered at the same dosage, a Tₘₐₓ not greater than about 90%, not greater than about 80%, not greater than about 70%, not greater than about 60%, not greater than about 50%, not greater than about 30%, not greater than about 25%, not greater than about 20%, not greater than about 15%, not greater than about 10%, or not greater than about 5% of the Tₘₐₓ exhibited by the non-nanoparticulate tadalafil formulation.

In another embodiment, the composition comprising at least one nanoparticulate tadalafil or a derivative or salt thereof, exhibits in comparative pharmacokinetic testing with a non-nanoparticulate formulation of the same tadalafil (e.g., Cialis), administered at the same dosage, a Cₘₐₓ which is at least about 50%, at least about 100%, at least about 200%, at least about 300%, at least about 400%, at least about 500%, at least about 600%, at least about 700%, at least about 800%, at least about 900%, at least about 1000%, at least about 1100%, at least about 1200%, at least about 1300%, at least about 1400%, at least about 1500%, at least about 1600%, at least about 1700%, at least about 1800%, or at least about 1900% greater than the Cₘₐₓ exhibited by the non-nanoparticulate tadalafil formulation.

In yet another embodiment, the composition comprising at least one nanoparticulate tadalafil or a derivative or salt thereof, exhibits in comparative pharmacokinetic testing with a non-nanoparticulate formulation of the same tadalafil (e.g., Cialis), administered at the same dosage, an AUC which is at least about 25%, at least about 50%, at least about 75%, at least about 100%, at least about 125%, at least about 150%, at least about 175%, at least about 200%, at least about 225%, at least about 250%, at least about 275%, at least about 300%, at least about 350%, at least about 400%, at least about 450%, at least about 500%, at least about 550%, at least about 600%, at least about 750%, at least about 700%, at least about 750%, at least about 800%, at least about 850%, at least about 900%, at least about 950%, at least about 1000%, at least about 1050%, at least about 1100%, at least about 1150%, or at least about 1200% greater than the AUC exhibited by the non-nanoparticulate tadalafil formulation.

3. The Pharmacokinetic Profiles of the Tadalafil Compositions are not Affected by the Fed or Fasted State of the Subject Ingesting the Compositions

In one embodiment of the invention, the pharmacokinetic profile of the nanoparticulate PDE5 inhibitor compositions, such as tadalafil, are not substantially affected by the fed or fasted state of a subject ingesting the composition. This means that there would be little or no appreciable difference in the quantity of drug absorbed or the rate of drug absorption when the nanoparticulate tadalafil compositions are administered in the fed or fasted state.

Benefits of a dosage form which substantially eliminates the effect of food include an increase in subject convenience, thereby increasing subject compliance, as the subject does not need to ensure that they are taking a dose either with or without food. This is significant, as with poor subject compliance an increase in the medical condition for which the drug is being prescribed may be observed.

4. Bioequivalency of Tadalafil Compositions When Administered in the Fed Versus the Fasted State

In one embodiment of the invention, administration of a nanoparticulate PDE5 inhibitor composition, such as tadalafil, to a subject in a fasted state is bioequivalent to administration of the composition to a subject in a fed state. The difference in absorption of the nanoparticulate tadalafil compositions, when administered in the fed versus the fasted state, preferably is less than about 100%, less than about 90%, less than about 80%, less than about 70%, less than about 60%, less than about 55%, less than about 50%, less than about 45%, less than about 40%, less than about 35%, less than about 30%, less than about 25%, less than about 20%, less than about 15%, less than about 10%, less than about 5%, or less than about 3%.

In some embodiments, the administration of the nanoparticulate tadalafil composition to a subject in a fasted state is bioequivalent to administration of the composition to a subject in a fed state, in particular as defined by Cₘₐₓ and AUC guidelines given by the U.S. Food and Drug Administration and the corresponding European regulatory agency (EMEA). Under U.S. FDA guidelines, two products or methods are bioequivalent if the 90% Confidence Intervals (CI) for AUC and Cₘₐₓ are between 0.80 to 1.25 (Tₘₐₓ measurements are not relevant to bioequivalence for regulatory purposes). To show bioequivalency between two compounds or administration conditions pursuant to Europe's EMEA guidelines, the 90% CI for AUC must be between 0.80 to 1.25 and the 90% CI for Cₘₐₓ must between 0.70 to 1.43.

5. Dissolution Profiles of the Tadalafil Compositions

The nanoparticulate PDE5 inhibitor compositions, such as tadalafil, are proposed to have unexpectedly dramatic dissolution profiles. Rapid dissolution of an administered active agent is preferable, as faster dissolution generally leads to faster absorption, onset of action and greater bioavailability. Additionally, a faster dissolution rate would allow for a larger dose of the drug to be absorbed, which would increase drug efficacy. To improve the dissolution profile and bioavailability of the tadalafil, it would be useful to increase the drug's dissolution so that it could attain a level close to 100%.

The tadalafil compositions of the invention are proposed to have a dissolution profile in which within about 5 minutes at least about 20% of the composition is dissolved. In other embodiments, at least about 30% or at least about 40% of the tadalafil composition is dissolved within about 5 minutes. In yet other embodiments, preferably at least about 40%, at least about 50%, at least about 60%, at least about 70%, or at least about 80% of the tadalafil composition is dissolved within about 10 minutes. In further embodiments, preferably at least about 70%, at least about 80%, at least about 90%, or at least about 100% of the tadalafil composition is dissolved within 20 minutes.

In some embodiments, dissolution is preferably measured in a medium which is discriminating. Such a dissolution medium will produce two very different dissolution curves for two products having very different dissolution profiles in gastric juices; i.e., the dissolution medium is predictive of in vivo dissolution of a composition. An exemplary dissolution medium is an aqueous medium containing the surfactant sodium lauryl sulfate at 0.025 M. Determination of the amount dissolved can be carried out by spectrophotometry. The rotating blade method (European Pharmacopoeia) can be used to measure dissolution.

6. Redispersibility of the Tadalafil Compositions of the Invention

An additional feature of the PDE5 inhibitor compositions, such as tadalafil, described herein may include redispersion such that the effective average particle size of the redispersed tadalafil particles is less than about 2 microns. This is significant, as if upon administration the tadalafil compositions of the invention did not redisperse to a substantially nanoparticulate size, then the dosage form may lose the benefits afforded by formulating the tadalafil into a nanoparticulate size.

Not wishing to be bound by any theory, it is proposed that nanoparticulate active agent compositions benefit from the small particle size of the active agent; if the active agent does not redisperse into the small particle sizes upon administration, then “clumps” or agglomerated active agent particles are formed, owing to the extremely high surface free energy of the nanoparticulate system and the thermodynamic driving force to achieve an overall reduction in free energy. With the formation of such agglomerated particles, the bioavailability of the dosage form may fall.

Moreover, the nanoparticulate tadalafil compositions of the invention are proposed to exhibit dramatic redispersion upon administration to a mammal, such as a human, as demonstrated by reconstitution/redispersion in a biorelevant aqueous media such that the effective average particle size of the redispersed tadalafil particles is less than about 2 microns. Such biorelevant aqueous media can be any aqueous media that exhibit the desired ionic strength and pH, which form the basis for the biorelevance of the media. In some embodiments, the desired pH and ionic strength are those that are representative of physiological conditions found in the human body. Such biorelevant aqueous media can be, for example, water, aqueous electrolyte solutions or aqueous solutions of any salt, acid, or base, or a combination thereof, which exhibit the desired pH and ionic strength. Such redispersion in a biorelevant media is predictive of in vivo efficacy of the tadalafil dosage form.

Biorelevant pH is well known in the art. For example, in the stomach, the pH ranges from slightly less than 2 (but typically greater than 1) up to 4 or 5. In the small intestine the pH can range from 4 to 6, and in the colon it can range from 6 to 8. Biorelevant ionic strength is also well known in the art. Fasted state gastric fluid has an ionic strength of about 0.1M while fasted state intestinal fluid has an ionic strength of about 0.14. See e.g., Lindahl et al., “Characterization of Fluids from the Stomach and Proximal Jejunum in Men and Women,” Pharm. Res., 14 (4): 497-502 (1997).

It is believed that the pH and ionic strength of the test solution is more critical than the specific chemical content. Accordingly, appropriate pH and ionic strength values can be obtained through numerous combinations of strong acids, strong bases, salts, single or multiple conjugate acid-base pairs (i.e., weak acids and corresponding salts of that acid), monoprotic and polyprotic electrolytes, etc.

Representative electrolyte solutions can be, but are not limited to, HCl solutions, ranging in concentration from about 0.001 to about 0.1 N, and NaCl solutions, ranging in concentration from about 0.001 to about 0.1 M, and mixtures thereof. For example, electrolyte solutions can be, but are not limited to, about 0.1 N HCl or less, about 0.01 N HCl or less, about 0.001 N HCl or less, about 0.1 M NaCl or less, about 0.01 M NaCl or less, about 0.001 M NaCl or less, and mixtures thereof. Of these electrolyte solutions, 0.01 M HCl and/or 0.1 M NaCl, are most representative of fasted human physiological conditions, owing to the pH and ionic strength conditions of the proximal gastrointestinal tract.

Electrolyte concentrations of 0.001 N HCl, 0.01 N HCl, and 0.1 N HCl correspond to pH 3, pH 2, and pH 1, respectively. Thus, a 0.01 N HCl solution simulates typical acidic conditions found in the stomach. A solution of 0.1 M NaCl provides a reasonable approximation of the ionic strength conditions found throughout the body, including the gastrointestinal fluids, although concentrations higher than 0.1 M may be employed to simulate fed conditions within the human GI tract.

Exemplary solutions of salts, acids, bases or combinations thereof, which exhibit the desired pH and ionic strength, include but are not limited to phosphoric acid/phosphate salts+sodium, potassium and calcium salts of chloride, acetic acid/acetate salts+sodium, potassium and calcium salts of chloride, carbonic acid/bicarbonate salts+sodium, potassium and calcium salts of chloride, and citric acid/citrate salts+sodium, potassium and calcium salts of chloride.

In other embodiments, the redispersed tadalafil particles (redispersed in water, a biorelevant medium, or any other suitable dispersion medium) have an effective average particle size of less than about less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods.

In still other embodiments, the redispersed tadalafil particles, when administered to a mammal, redisperse such that the particles have an effective average particle size of less than about 2000 nm, less than about 1900 run, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods.

Redispersibility can be tested using any suitable means known in the art. See e.g., the example sections of U.S. Pat. No. 6,375,986 for “Solid Dose Nanoparticulate Compositions Comprising a Synergistic Combination of a Polymeric Surface Stabilizer and Dioctyl Sodium Sulfosuccinate.”

7. Tadalafil Compositions Used in Conjunction with other Active Agents

The compositions comprising nanoparticulate PDE5 inhibitor, such as tadalafil or salts or derivatives thereof, can additionally include one or more compounds useful in the treatment of sexual dysfunction, erectile dysfunction and related disorders. Examples of such compounds include, but are not limited to one or more of other PDE5 inhibitors such as sildenafil and vardenafil; testosterone; bremelanotide (formerly known as PT-141); ginseng and combinations thereof.

C. Nanoparticulate Tadalafil Compositions

The invention provides compositions comprising PDE5 inhibitors, such as tadalafil particles and at least one surface stabilizer. The surface stabilizers preferably are adsorbed on, or associated with, the surface of the tadalafil particles. In some embodiments, surface stabilizers preferably physically adhere on, or associate with, the surface of the nanoparticulate tadalafil particles, but do not chemically react with the tadalafil particles or itself. Individually adsorbed molecules of the surface stabilizer are essentially free of intermolecular cross-linkages.

The present invention also includes tadalafil compositions together with one or more non-toxic physiologically acceptable carriers, adjuvants, or vehicles, collectively referred to as carriers. The compositions can be formulated for parenteral injection (e.g., intravenous, intramuscular, or subcutaneous), oral administration in solid, liquid, or aerosol form, vaginal, nasal, rectal, ocular, local (powders, ointments or drops), buccal, intracistemal, intraperitoneal, or topical administration, and the like.

1. Tadalafil Particles

The compositions of the invention comprise particles of tadalafil or a salt or derivative thereof. The particles can be in crystalline phase, semi-crystalline phase, amorphous phase, semi-amorphous phase, or a combination thereof.

2. Surface Stabilizers

The choice of a surface stabilizer for a tadalafil is non-trivial and required extensive experimentation to realize a desirable formulation. Accordingly, the present invention is directed to the surprising discovery that nanoparticulate tadalafil compositions can be made.

Combinations of more than one surface stabilizers may be used in the invention. Suitable surface stabilizers which can be employed in the invention include, but are not limited to, known organic and inorganic pharmaceutical excipients. Such excipients include various polymers, low molecular weight oligomers, natural products, and surfactants. Surface stabilizers include nonionic, anionic, cationic, ionic, and zwitterionic surfactants.

Representative examples of surface stabilizers include hydroxypropyl methylcellulose (now known as hypromellose), hydroxypropylcellulose, polyvinylpyrrolidone, sodium lauryl sulfate, dioctylsulfosuccinate (dioctyl sodium sulfosuccinate), gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween® 20 and Tween® 80 (ICI Speciality Chemicals)); polyethylene glycols (e.g., Carbowaxs® 3550 and 934 (Union Carbide)), polyoxyethylene stearates, colloidal silicon dioxide, phosphates, carboxymethylcellulose calcium, carboxymethylcellulose sodium, methylcellulose, hydroxyethylcellulose, hypromellose phthalate, noncrystalline cellulose, magnesium aluminium silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics® F68 and F108, which are block copolymers of ethylene oxide and propylene oxide); poloxamines (e.g., Tetronic® 908, also known as Poloxamine™ 908, which is a tetrafunctional block copolymer derived from sequential addition of propylene oxide and ethylene oxide to ethylenediamine (BASF Wyandotte Corporation, Parsippany, N.J.)); Tetronic® 1508 (T-1508) (BASF Wyandotte Corporation), Tritons® X-200, which is an alkyl aryl polyether sulfonate (Rohm and Haas); Crodestas™ F-110, which is a mixture of sucrose stearate and sucrose distearate (Croda Inc.); p-isononylphenoxypoly-(glycidol), also known as Olin®-lOG or Surfactant™ 10-G (Olin Chemicals, Stamford, Conn.); Crodestas™ SL-40 (Croda, Inc.); and SA9OHCO, which is C₁₈H₃₇CH₂(CON(CH₃)—CH₂(CHOH)₄(CH₂0H)₂ (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; PEG-phospholipid, PEG-cholesterol, PEG-cholesterol derivative, PEG-vitamin A, PEG-vitamin E, lysozyme, random copolymers of vinyl pyrrolidone and vinyl acetate, and the like.

Examples of useful cationic surface stabilizers include, but are not limited to, polymers, biopolymers, polysaccharides, cellulosics, alginates, phospholipids, and nonpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), hexyldesyltrimethylammonium bromide (HDMAB), and polyvinylpyrrolidone-2-dimethylaminoethyl methacrylate dimethyl sulfate.

Other useful cationic stabilizers include, but are not limited to, cationic lipids, sulfonium, phosphonium, and quartemary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C₁₂₋₁₅dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl (ethenoxy)₄ ammonium chloride or bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₅, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336™), POLYQUAT 10™, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quaternized polyoxyethylalkylamines, MIRAPOL™ and ALKAQUAT™ (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar.

Such exemplary cationic surface stabilizers and other useful cationic surface stabilizers are described in J. Cross and E. Singer, Cationic Surfactants: Analytical and Biological Evaluation (Marcel Dekker, 1994); P. and D. Rubingh (Editor), Cationic Surfactants: Physical Chemistry (Marcel Dekker, 1991); and J. Richmond, Cationic Surfactants: Organic Chemistry, (Marcel Dekker, 1990).

Nonpolymeric surface stabilizers are any nonpolymeric compound, such benzalkonium chloride, a carbonium compound, a phosphonium compound, an oxonium compound, a halonium compound, a cationic organometallic compound, a quartemary phosphorous compound, a pyridinium compound, an anilinium compound, an ammonium compound, a hydroxylammonium compound, a primary ammonium compound, a secondary ammonium compound, a tertiary ammonium compound, and quartemary ammonium compounds of the formula NR₁R₂R₃R₄⁽⁺⁾. For compounds of the formula NR₁R₂R₃R₄⁽⁺⁾:
(i) none of R₁-R₄ are CH₃;
(ii) one of R₁-R₄ is CH₃;
(iii) three of R₁-R₄ are CH₃;
(iv) all of R₁-R₄ are CH₃;
(v) two of R₁-R₄ are CH₃, one of R₁-R₄ is C₆H₅CH₂, and one of R₁-R₄ is an alkyl chain of seven carbon atoms or less;
(vi) two of R₁-R₄ are CH₃, one of R₁-R₄ is C₆H₅CH₂, and one of R₁-R₄ is an alkyl chain of nineteen carbon atoms or more;
(vii) two of R₁-R₄ are CH₃ and one of R₁-R₄ is the group C₆H₅(CH₂)ₙ, where n>1;
(viii) two of R₁-R₄ are CH₃, one of R₁-R₄ is C₆H₅CH₂, and one of R₁-R₄ comprises at least one heteroatom;
(ix) two of R₁-R₄ are CH₃, one of R₁-R₄ is C₆H₅CH₂, and one of R₁-R₄ comprises at least one halogen;
(x) two of R₁-R₄ are CH₃, one of R₁-R₄ is C₆H₅CH₂, and one of R₁-R₄ comprises at least one cyclic fragment;
(xi) two of R₁-R₄ are CH₃ and one of R₁-R₄ is a phenyl ring; or
(xii) two of R₁-R₄ are CH₃ and two of R₁-R₄ are purely aliphatic fragments.

Such compounds include, but are not limited to, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride(Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

In some embodiments, the surface stabilizers may include copovidone (e.g., Plasdone S630, which is random copolymer of vinyl acetate and vinyl pyrrolidone) and docusate sodium.

In other embodiments, the surface stabilizer may include a povidone polymer. Povidone polymers are exemplary surface stabilizers that could be used in formulating an injectable nanoparticulate tadalafil composition. Povidone polymers, also known as polyvidon(e), povidonum, PVP, and polyvinylpyrrolidone, are sold under the trade names Kollidon® (BASF Corp.) and Plasdone® (ISP Technologies, Inc.). They are polydisperse macromolecular molecules, with a chemical name of 1-ethenyl-2-pyrrolidinone polymers and 1-vinyl-2-pyrrolidinone polymers. Povidone polymers are produced commercially as a series of products having mean molecular weights ranging from about 10,000 to about 700,000 daltons. In some embodiments, the povidone polymer may have a molecular weight of less than about 40,000 daltons, as a molecular weight of greater than 40,000 daltons could have difficulty clearing the body of a mammal.

Povidone polymers are prepared by, for example, Reppe's process, comprising: (1) obtaining 1,4-butanediol from acetylene and formaldehyde by the Reppe butadiene synthesis; (2) dehydrogenating the 1,4-butanediol over copper at 200° to form y-butyrolactone; and (3) reacting y-butyrolactone with ammonia to yield pyrrolidone. Subsequent treatment with acetylene gives the vinyl pyrrolidone monomer. Polymerization is carried out by heating in the presence of H₂O and NH₃. See The Merck Index, 10th Edition, pp. 7581 (Merck & Co., Rahway, N.J., 1983).

The manufacturing process for povidone polymers produces polymers containing molecules of unequal chain length, and thus different molecular weights. The molecular weights of the molecules vary about a mean or average for each particular commercially available grade. Because it is difficult to determine the polymer's molecular weight directly, the most widely used method of classifying various molecular weight grades is by K-values, based on viscosity measurements. The K-values of various grades of povidone polymers represent a function of the average molecular weight, and are derived from viscosity measurements and calculated according to Fikentscher's formula.

The weight-average of the molecular weight, Mw, is determined by methods that measure the weights of the individual molecules, such as by light scattering. Table 1 provides molecular weight data for several commercially available povidone polymers, all of which are soluble.

TABLE 1

| | | Mv | Mw | Mn |
|---|---|---|---|---|
| Povidone | K-Value | (Daltons)** | (Daltons)** | (Daltons)** |
| Plasdone | 17 ± 1 | 7,000 | 10,500 | 3,000 |
| C-15 ® | | | | |
| Plasdone | 30.5 ± 1.5 | 38,000 | 62,500* | 16,500 |
| C-30 ® | | | | |
| Kollidon 12 | 11-14 | 3,900 | 2,000-3,000 | 1,300 |
| PF ® | | | | |
| Kollidon 17 | 16-18 | 9,300 | 7,000-11,000 | 2,500 |
| PF ® | | | | |
| Kollidon | 24-32 | 25,700 | 28,000-34,000 | 6,000 |
| 25 ® | | | | |

*Because the molecular weight is greater than 40,000 daltons, this povidone polymer may not be useful as a surface stabilizer for a drug compound to be administered parenterally (i.e., injected).
**Mv is the viscosity-average molecular weight, Mn is the number-average molecular weight, and Mw is the weight average molecular weight. Mw and Mn were determined by light scattering and ultra-centrifugation, and Mv was determined by viscosity measurements.

Based on the data provided in Table 1, exemplary commercially available povidone polymers that may be used in some embodiments include, but are not limited to, Plasdone C-15®, Kollidon 12 PF®, Kollidon 17 PF®, and Kollidon 25®.

Many surface stabilizers are commercially available and/or can be prepared by techniques known in the art. See e.g., Handbook of pharmaceutical Excipients, published jointly by the American Pharmaceutical Association and The Pharmaceutical Society of Great Britain (The Pharmaceutical Press, 2000), specifically incorporated by reference.

3. Other Pharmaceutical Excipients

Pharmaceutical compositions according to the invention may also comprise one or more binding agents, filling agents, lubricating agents, suspending agents, sweeteners, flavoring agents, preservatives, buffers, wetting agents, disintegrants, effervescent agents, and other excipients. Such excipients are known in the art.

Examples of filling agents include lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™).

Suitable lubricants, including agents that act on the flowability of the powder to be compressed, include colloidal silicon dioxide, such as Aerosil® 200, talc, stearic acid, magnesium stearate, calcium stearate, and silica gel.

Examples of sweeteners include any natural or artificial sweetener, such as sucrose, xylitol, sodium saccharin, cyclamate, aspartame, and acsulfame. Examples of flavoring agents include Magnasweete® (trademark of MAFCO), bubble gum flavor, and fruit flavors, and the like.

Examples of preservatives include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of buffers include phosphate buffer, citrate buffers and buffers made from other organic acids.

Examples of wetting or dispersing agents include a naturally-occurring phosphatide, for example, lecithin or condensation products of n-alkylene oxide with fatty acids, for example, polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol mono-oleate, or condensation products of ethylene oxide with partial esters derived from fatty acids and hexitol anhydrides, for example, polyethylene sorbitan monooleate.

Examples of effervescent agents include effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

4. Nanoparticulate Tadalafil Particle Size

The compositions of the invention comprise nanoparticulate PDE5 inhibitors, such as tadalafil, which have an effective average particle size of less than about 2000 nm (i.e., 2 microns), less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods.

By “an effective average particle size of less than about 2000 nm” it is meant that at least 50% of the tadalafil particles have a particle size of less than the effective average, by weight (or by other suitable measurement technique, such as by volume, number, etc.), i.e., less than about 2000 nm, less than about 1900 nm, less than about 1800 nm, etc., when measured by techniques known in the art, such as those noted above. In some embodiments, at least about 70%, at least about 90%, or at least about 95% of the tadalafil particles have a particle size of less than the effective average, i.e., less than about 2000 nm, less than about 1900 nm, less than about 1800 nm, less than about 1700 nm, etc.

As used herein, the value for D50 of a nanoparticulate tadalafil composition is the particle size below which 50% of the tadalafil particles fall, by weight (or by other suitable measurement technique, such as by volume, number, etc.). Similarly, D90 is the particle size below which 90% of the tadalafil particles fall, by weight (or by other suitable measurement technique, such as by volume, number, etc.).

5. Concentration of Tadalafil and Surface Stabilizers

The relative amounts of tadalafil, or a salt or derivative thereof, and one or more surface stabilizers may vary. The optimal amount of the individual components can depend, for example, upon the particular tadalafil selected, the hydrophilic lipophilic balance (HLB), melting point, and the surface tension of water solutions of the stabilizer, etc.

In some embodiments, the concentration of the tadalafil may vary from about 99.5% to about 0.001%, from about 95% to about 0.1%, or from about 90% to about 0.5%, by weight, based on the total combined dry weight of the tadalafil and at least one surface stabilizer, not including other excipients.

In other embodiments, the concentration of the at least one surface stabilizer may vary from about 0.5% to about 99.999%, from about 5.0% to about 99.9%, or from about 10% to about 99.5%, by weight, based on the total combined dry weight of the tadalafil and at least one surface stabilizer, not including other excipients.

6. Exemplary Nanoparticulate Tadalafil Tablet Formulations

Several exemplary tadalafil tablet formulations are given below. These examples are not intended to limit the invention in any respect, but rather to provide exemplary tablet formulations of tadalafil which can be used as described herein and by methods known in the art. Such exemplary tablets may also comprise a coating agent.

Exemplary Nanoparticulate
Tadalafil Tablet Formulation #1

| Component | g/Kg |
|---|---|
| Tadalafil | about 50 to about 500 |
| Hypromellose, USP | about 10 to about 70 |
| Docusate Sodium, USP | about 1 to about 10 |
| Sucrose, NF | about 100 to about 500 |
| Sodium Lauryl Sulfate, NF | about 1 to about 40 |
| Lactose Monohydrate, NF | about 50 to about 400 |
| Silicified Microcrystalline Cellulose | about 50 to about 300 |
| Crospovidone, NF | about 20 to about 300 |
| Magnesium Stearate, NF | about 0.5 to about 5 |

Exemplary Nanoparticulate
Tadalafil Tablet Formulation #2

| Component | g/Kg |
|---|---|
| Tadalafil | about 100 to about 300 |
| Hypromellose, USP | about 30 to about 50 |
| Docusate Sodium, USP | about 0.5 to about 10 |
| Sucrose, NF | about 100 to about 300 |
| Sodium Lauryl Sulfate, NF | about 1 to about 30 |
| Lactose Monohydrate, NF | about 100 to about 300 |
| Silicified Microcrystalline Cellulose | about 50 to about 200 |
| Crospovidone, NF | about 50 to about 200 |
| Magnesium Stearate, NF | about 0.5 to about 5 |

Exemplary Nanoparticulate
Tadalafil Tablet Formulation #3

| Component | g/Kg |
|---|---|
| Tadalafil | about 200 to about 225 |
| Hypromellose, USP | about 42 to about 46 |
| Docusate Sodium, USP | about 2 to about 6 |
| Sucrose, NF | about 200 to about 225 |
| Sodium Lauryl Sulfate, NF | about 12 to about 18 |
| Lactose Monohydrate, NF | about 200 to about 205 |
| Silicified Microcrystalline Cellulose | about 130 to about 135 |
| Crospovidone, NF | about 112 to about 118 |
| Magnesium Stearate, NF | about 0.5 to about 3 |

Exemplary Nanoparticulate
Tadalafil Tablet Formulation #4

| Component | g/Kg |
|---|---|
| Tadalafil | about 119 to about 224 |
| Hypromellose, USP | about 42 to about 46 |
| Docusate Sodium, USP | about 2 to about 6 |
| Sucrose, NF | about 119 to about 224 |
| Sodium Lauryl Sulfate, NF | about 12 to about 18 |
| Lactose Monohydrate, NF | about 119 to about 224 |
| Silicified Microcrystalline Cellulose | about 129 to about 134 |
| Crospovidone, NF | about 112 to about 118 |
| Magnesium Stearate, NF | about 0.5 to about 3 |

7. Injectable Nanoparticulate Tadalafil Formulations

In some embodiments, injectable nanoparticulate tadalafil formulations are provided. The following example is not intended to limit the scope of nanoparticulate injectable formulations in any respect, but rather to provide exemplary formulations which can be utilized as described herein and by methods known in the art. In some embodiments, the injectable formulations may comprise high drug concentrations in low injection volumes. Further, duration of action may be controlled via manipulation of particle size and hence dissolution, resulting in efficacious blood levels for extended periods; for example, greater than 2 days, greater than 5 days, greater than 7 days, greater than 10 days or greater than 14 days. An illustrative, non-limiting compositions is described below (based on % w/w):

| | |
|---|---|
| Tadalafil | 5-50% |
| Stabilizer polymer | 0.1-50% |
| preservatives (Optional) | 0.05-0.25% |
| pH adjusting agent | pH about 6 to about 7 |
| water for injection | q.s. |

Exemplary preservatives include methylparaben (about 0.18% based on % w/w), propylparaben (about 0.02% based on % w/w), phenol (about 0.5% based on % w/w), and benzyl alcohol (up to 2% v/v). An exemplary pH adjusting agent is sodium hydroxide, and an exemplary liquid carrier is sterile water for injection. Other useful preservatives, pH adjusting agents, and liquid carriers are well-known in the art.

Exemplary surface stabilizers for injectable tadalafil formulations may include but are not limited to stabilizers such as povidone polymer, hydroxypropyl cellulose, hydroxypropyl methyl cellulose, providone, polyvinyl pyrrolidone (PVP), pluronics, Tween®, peg-phospholipids and mixtures thereof. In some embodiments, stabilizers such as povidone, with a molecular weight of less than about 40,000 daltons, may be preferred. These stabilizers may be adsorbed onto the surface of the tadalafil particle in an amount sufficient to maintain an effective average particle size for the desired duration of efficacy. Further, the nanoparticle size can be manipulated to give the desirable blood level profiles and duration of action when administered by either IM or SC routes.

D. Methods of Making Nanoparticulate Tadalafil Compositions

The nanoparticulate PDE5 inhibitor compositions, such as nanoparticulate tadalafil compositions, can be made using, for example, milling, homogenization, precipitation, freezing, supercritical particle generation, or template emulsion techniques. Exemplary methods of making nanoparticulate compositions are described in the '684 patent. Methods of making nanoparticulate active agent compositions are also described in U.S. Pat. No. 5,518,187 for “Method of Grinding Pharmaceutical Substances”; U.S. Pat. No. 5,718,388 for “Continuous Method of Grinding Pharmaceutical Substances”; U.S. Pat. No. 5,862,999 for “Method of Grinding Pharmaceutical Substances”; U.S. Pat. No. 5,665,331 for “Co-Microprecipitation of Nanoparticulate Pharmaceutical Agents with Crystal Growth Modifiers”; U.S. Pat. No. 5,662,883 for “Co-Microprecipitation of Nanoparticulate Pharmaceutical Agents with Crystal Growth Modifiers”; U.S. Pat. No. 5,560,932 for “Microprecipitation of Nanoparticulate Pharmaceutical Agents”; U.S. Pat. No. 5,543,133 for “Process of Preparing X-Ray Contrast Compositions Containing Nanoparticles”; U.S. Pat. No. 5,534,270 for “Method of Preparing Stable Drug Nanoparticles”; U.S. Pat. No. 5,510,118 for “Process of Preparing Therapeutic Compositions Containing Nanoparticles”; and U.S. Pat. No. 5,470,583 for “Method of Preparing Nanoparticle Compositions Containing Charged Phospholipids to Reduce Aggregation,” all of which are specifically incorporated by reference.

The resultant nanoparticulate tadalafil compositions or dispersions can be utilized in solid or liquid dosage formulations, such as liquid dispersions, gels, aerosols, ointments, creams, controlled release formulations, fast melt formulations, lyophilized formulations, tablets, capsules, delayed release formulations, extended release formulations, pulsatile release formulations, mixed immediate release and controlled release formulations, etc.

1. Milling to Obtain Nanoparticulate Tadalafil Dispersions

Milling a tadalafil, or a salt or derivative thereof, to obtain a nanoparticulate dispersion comprises dispersing the tadalafil particles in a liquid dispersion medium in which the tadalafil is poorly soluble, followed by applying mechanical means in the presence of grinding media to reduce the particle size of the tadalafil to the desired effective average particle size. The dispersion medium can be, for example, water, safflower oil, ethanol, t-butanol, glycerin, polyethylene glycol (PEG), hexane, or glycol. In some embodiments, a preferred dispersion medium is water.

The tadalafil particles can be reduced in size in the presence of at least one surface stabilizer. Alternatively, tadalafil particles can be contacted with one or more surface stabilizers after attrition. Other compounds, such as a diluent, can be added to the tadalafil/surface stabilizer composition during the size reduction process. Dispersions can be manufactured continuously or in a batch mode.

2. Precipitation to Obtain Nanoparticulate Tadalafil Compositions

Another method of forming the desired nanoparticulate tadalafil compositions is by microprecipitation. This is a method of preparing stable dispersions of poorly soluble active agents in the presence of one or more surface stabilizers and one or more colloid stability enhancing surface active agents free of any trace toxic solvents or solubilized heavy metal impurities. Such a method comprises, for example: (1) dissolving the tadalafil in a suitable solvent; (2) adding the formulation from step (1) to a solution comprising at least one surface stabilizer; and (3) precipitating the formulation from step (2) using an appropriate non-solvent. The method can be followed by removal of any formed salt, if present, by dialysis or diafiltration and concentration of the dispersion by conventional means.

3. Homogenization to Obtain Nanoparticulate Tadalafil Compositions

Exemplary homogenization methods of preparing active agent nanoparticulate compositions are described in U.S. Pat. No. 5,510,118, for “Process of Preparing Therapeutic Compositions Containing Nanoparticles.” Such a method comprises dispersing particles of a tadalafil, or a salt or derivative thereof, in a liquid dispersion medium, followed by subjecting the dispersion to homogenization to reduce the particle size of a tadalafil to the desired effective average particle size. The tadalafil particles can be reduced in size in the presence of at least one surface stabilizer. Alternatively, the tadalafil particles can be contacted with one or more surface stabilizers either before or after attrition. Other compounds, such as a diluent, can be added to the tadalafil/surface stabilizer composition either before, during, or after the size reduction process. Dispersions can be manufactured continuously or in a batch mode.

4. Cryogenic Methodologies to Obtain Nanoparticulate Tadalafil Compositions

Another method of forming the desired nanoparticulate tadalafil compositions is by spray freezing into liquid (“SFL”). This technology comprises an organic or organoaqueous solution of tadalafil with stabilizers, which is injected into a cryogenic liquid, such as liquid nitrogen. The droplets of the tadalafil solution freeze at a rate sufficient to minimize crystallization and particle growth, thus formulating nanostructured tadalafil particles. Depending on the choice of solvent system and processing conditions, the nanoparticulate tadalafil particles can have varying particle morphology. In the isolation step, the nitrogen and solvent are removed under conditions that avoid agglomeration or ripening of the tadalafil particles.

As a complementary technology to SFL, ultra rapid freezing (“URF”) may also be used to created equivalent nanostructured tadalafil particles with greatly enhanced surface area.

URF comprises an organic or organoaqueous solution of tadalafil with stabilizers onto a cryogenic substrate.

5. Emulsion Methodologies to Obtain Nanoparticulate Tadalafil Compositions

Another method of forming the desired nanoparticulate tadalafil, or a salt or derivative thereof, composition is by template emulsion. Template emulsion creates nanostructured tadalafil particles with controlled particle size distribution and rapid dissolution performance. The method comprises an oil-in-water emulsion that is prepared, then swelled with a non-aqueous solution comprising the tadalafil and stabilizers. The particle size distribution of the tadalafil particles is a direct result of the size of the emulsion droplets prior to loading with the tadalafil, a property which can be controlled and optimized in this process. Furthermore, through selected use of solvents and stabilizers, emulsion stability is achieved with no or suppressed Ostwald ripening. Subsequently, the solvent and water are removed, and the stabilized nanostructured tadalafil particles are recovered. Various tadalafil particle morphologies can be achieved by appropriate control of processing conditions.

6. Supercritical Fluid Techniques Used to Obtain Nanoparticulate Tadalafil Compositions

Published International Patent Application No. WO 97/14407 to Pace et al., published Apr. 24, 1997, discloses particles of water insoluble biologically active compounds with an average size of 100 nm to 300 nm that are prepared by dissolving the compound in a solution and then spraying the solution into compressed gas, liquid or supercritical fluid in the presence of appropriate surface modifiers.

7. Sterile Product Manufacturing

Development of injectable compositions requires the production of a sterile product. The manufacturing process of the present invention is similar to typical known manufacturing processes for sterile suspensions. A typical sterile suspension manufacturing process flowchart is as follows:

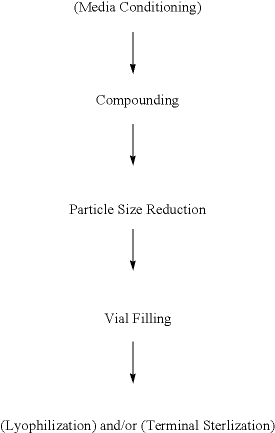

As indicated by the optional steps in parentheses, some of the processing is dependent upon the method of particle size reduction and/or method of sterilization. For example, media conditioning is not required for a milling method that does not use media. If terminal sterilization is not feasible due to chemical and/or physical instability, aseptic processing can be used.

E. Methods of Using the Nanoparticulate Tadalafil Compositions of the Invention

The invention provides a method of rapidly increasing the bioavailability (e.g., plasma levels) of PDE5 inhibitors, such as tadalafil, in a subject. Such a method comprises orally administering to a subject an effective amount of a composition comprising a PDE5 inhibitor (e.g., tadalafil) in nanoparticulate form. In some embodiments, the tadalafil compositions, in accordance with standard pharmacokinetic practice, have a bioavailability that is about 50% greater, about 40% greater, about 30% greater, about 20% greater or about 10% greater than a conventional dosage form. Additionally, when tested in fasting subjects in accordance with standard pharmacokinetic practice, the nanoparticulate tadalafil compositions produce a maximum blood plasma concentration profile in less than about 6 hours, less than about 5 hours, less than about 4 hours, less than about 3 hours, less than about 2 hours, less than about 1 hour, or less than about 30 minutes after the initial dose of the compositions.

The invention also provides compositions which are proposed to have faster absorption and a faster onset of therapeutic effect than conventional formulations of the same drug. The compositions of the invention are proposed to be useful in the treatment of sexual dysfunction such as erectile dysfunction, and vascular disorders or diseases such as pulmonary arterial hypertension, the effects and symptoms of myocardial infarction, ischemia/reperfusion injury, inflammatory and degenerative lung disorders, for example, chronic obstructive pulmonary disease (COPD), adult respiratory distress syndrome (ARDS), acute lung injury (ALI), bronchitis, bronchial asthma, pulmonary fibroses, emphysema, interstitial pulmonary disorders and pneumonias when administered to a subject in need of such treatment.

As such, some methods include administering a composition comprising a nanoparticulate tadalafil and at least one surface stabilizer.

The tadalafil compounds of the invention can be administered to a subject via any conventional means including, but not limited to, orally, rectally, ocularly, parenterally (e.g., intravenous, intramuscular, or subcutaneous), intracisternally, pulmonary, intravaginally, intraperitoneally, locally (e.g., powders, ointments or drops), as a bioadhesive, or as a buccal or nasal spray.

In some embodiments, a solid dosage form may be preferred. Solid dosage forms for oral administration include, but are not limited to, capsules, tablets, pills, powders, and granules. In such solid dosage forms, the active agent may be admixed with at least one of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, or mixtures thereof. For capsules, tablets, and pills, the dosage forms may also comprise buffering agents.

Liquid dosage forms for oral administration may include pharmaceutically acceptable emulsions, solutions, suspensions, syrups, and elixirs. In addition to a PDE5 inhibitor such as tadalafil, the liquid dosage forms may also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures of these substances, and the like.

Instead of or in addition to such inert diluents, the composition may also include adjuvants, such as wetting agents, emulsifying and suspending agents, sweetening, flavoring, and perfuming agents.

The tadalafil compositions may be formulated for parenteral administration; the nanoparticulate formulations would likely eliminate the need for toxic co-solvents and enhance the efficacy of tadalafil in the treatment of sexual dysfunction, such as erectile dysfunction, and vascular disorders or diseases such as pulmonary arterial hypertension, the effects and symptoms of myocardial infarction, ischemia/reperfusion injury, inflammatory and degenerative lung disorders, for example, chronic obstructive pulmonary disease (COPD), adult respiratory distress syndrome (ARDS), acute lung injury (ALI), bronchitis, bronchial asthma, pulmonary fibroses, emphysema, interstitial pulmonary disorders and pneumonias. Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, or vehicles including water, ethanol, polyols (propyleneglycol, polyethylene-glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

The nanoparticulate tadalafil, or a salt or derivative thereof, compositions may also contain adjuvants such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

“Therapeutically effective amount” as used herein with respect to a tadalafil, dosage shall mean that dosage that provides the specific pharmacological response for which tadalafil is administered in a significant number of subjects in need of such treatment. It is emphasized that “therapeutically effective amount,” administered to a particular subject in a particular instance will not always be effective in treating the diseases described herein, even though such dosage is deemed a “therapeutically effective amount” by those skilled in the art. It is to be further understood that tadalafil dosages are, in particular instances, measured as oral dosages, or with reference to drug levels as measured in blood.

One of ordinary skill will appreciate that effective amounts of a nanoparticulate tadalafil can be determined empirically and can be employed in pure form or, where such forms exist, in pharmaceutically acceptable salt, ester, or prodrug form. Actual dosage levels of a tadalafil in the nanoparticulate compositions of the invention may be varied to obtain an amount of a tadalafil that is effective to obtain a desired therapeutic response for a particular composition and method of administration. The selected dosage level therefore depends upon the desired therapeutic effect, the route of administration, the potency of the administered tadalafil, the desired duration of treatment, and other factors.

Dosage unit compositions may contain such amounts of such submultiples thereof as may be used to make up the daily dose. It will be understood, however, that the specific dose level for any particular patient will depend upon a variety of factors: the type and degree of the cellular or physiological response to be achieved; activity of the specific agent or composition employed; the specific agents or composition employed; the age, body weight, general health, sex, and diet of the patient; the time of administration, route of administration, and rate of excretion of the agent; the duration of the treatment; drugs used in combination or coincidental with the specific agent; and like factors well known in the medical arts.

### F. EXAMPLES

The following examples are given to illustrate the present invention. It should be understood, however, that the invention is not to be limited to the specific conditions or details described in these examples. Throughout the specification, any and all references to a publicly available document, including U.S. patents, are specifically incorporated by reference.

### Example 1

The purpose of this example is to demonstrate the preparation of compositions comprising nanoparticulate tadalafil or a salt or derivative thereof.

Eleven different formulations of nanoparticulate tadalafil were prepared using a NanoMill® 0.01, 10-ml chamber (NanoMill Systems, King of Prussia, Pa.; see e.g., U.S. Pat. No. 6,431,478) and 500-micron PolyMill® attrition media (Dow Chemical Co.), at a media load of about 89%. Each formulation was milled at 2500 rpm for 60 minutes, although mill speed and milling time may be varied (e.g., 2000-3500 RPM for 30-90 minutes) to determine optimal milling conditions for different formulations. The formulations are presented in Table 2.

Following milling, the tadalafil particles were evaluated using a Lecia DM5000B microscope and Lecia CTR 5000 light source (Laboratory Instruments & Supplies (I) Ltd. Ashboume Company, Meath, Ireland). Microscopy observations for each formulation are shown in Table 3 (note that no microscopy was performed on sample 8). Additionally, the particle size of the milled tadalafil particles was measured, using deionized, distilled water and a Horiba LA 910 particle size analyzer. After particle size analysis, a “successful composition” may define formulations in which the initial mean and/or D50 of milled tadalafil particle size is less than about 2000 nm. Particles were additionally analyzed before (“N”) and after (“Y”) a 60 second sonication. Table 4 shows the results of particle size analysis for each sample formulation, and Table 5 provides an evaluation of “successful formulation,” the basis of the evaluation, and comments regarding particle size analysis.

TABLE 2

| Sample | Tadalafil Formulations |
|---|---|
| 1 | Tadalafil, 5.00% w/w |
| | HPC-SL, 2.00% w/w |
| | Deionised Water, 93.00% w/w |
| 2 | Tadalafil, 5.00% w/w |
| | Plasdone K29/32, 1.25% w/w |
| | Sodium Lauryl Sulfate, 0.05% w/w |
| | Deionised Water, 93.70% w/w |
| 3 | Tadalafil, 5.00% w/w |
| | Pharmacoat 603, 1.25% w/w |
| | Docusate Sodium, 0.05% w/w |
| | Deionised Water, 93.70% w/w |
| 4 | Tadalafil, 5.00% w/w |
| | Tyloxapol, 1.00% w/w |
| | Deionised Water, 94.00% w/w |
| 5 | Tadalafil, 5.00% w/w |
| | Plasdone C15, 1.25% w/w |
| | Deoxycholate Sodium, 0.05% w/w |
| | Deionised Water, 93.70% w/w |
| 6 | Tadalafil, 5.00% w/w |
| | HPC-SL, 1.25% w/w |
| | Sodium Lauryl Sulfate, 0.05% w/w |
| | Deionised Water, 93.70% w/w |
| 7 | Tadalafil, 5.00% w/w |
| | Plasdone S-630, 1.25% w/w |
| | Docusate Sodium, 0.05% w/w |
| | Deionised Water, 93.70% w/w |
| 8 | Tadalafil, 5.00% w/w |
| | Plasdone C30, 1.25% w/w |
| | Docusate Sodium, 0.05% w/w |
| | Deionised Water, 93.70% w/w |
| 9 | Tadalafil, 5.00% w/w |
| | Lutrol F127, 1.25% w/w |
| | Sodium Lauryl Sulfate, 0.05% w/w |
| | Deionised Water, 93.70% w/w |
| 10 | Tadalafil, 5.00% w/w |
| | Lutrol F127, 1.00% w/w |
| | Tween 80, 1.00% w/w |
| | Deionised Water, 93.00% w/w |
| 11 | Tadalafil, 5.00% w/w |
| | Tween 80, 1.25% w/w |
| | Lecithin, 0.05% w/w |
| | Deionised Water, 93.70% w/w |

TABLE 3

| Sample | Microscopy observations |
|---|---|
| 1 | The sample exhibited severe flocculation over the whole |
| | surface of the slide. No Brownian motion was observed. |
| 2 | Microscopy showed the sample to be well dispersed with |
| | nanoparticles clearly visible. Acicular crystals were |
| | observed, most of which appeared less than 2000 nm. |
| 3 | Microscopy showed the sample to be well dispersed with |
| | nanoparticles clearly visible which exhibited Brownian |
| | motion. Acicular crystals were observed along with |
| | small numbers of unmilled particulates. |
| 4 | Mild flocculation was observed in the sample. Brownian |
| | motion was initially observed but the sample appeared to |
| | solidify after a very short time on the slide. A small |
| | number of acicular crystals were also observed. |
| 5 | No Brownian motion observed. Severe flocculation |
| | present. Acicular crystals were also present which may |
| | indicate crystal growth. |
| 6 | Microscopy showed the sample to be well dispersed with |
| | nanoparticles clearly visible. The particles exhibited |
| | Brownian motion. Relatively small numbers of larger |
| | acicular crystals were observed. These are possible |
| | resulting from crystal growth or unmilled material. |
| 7 | Microscopy showed the sample to be well dispersed with |
| | nanoparticles clearly visible. The particles exhibited |
| | Brownian motion. Relatively small numbers of larger |
| | acicular crystals were observed. These are possible |
| | resulting from crystal growth or unmilled material. |
| 8 | N/A |
| 9 | Microscopy showed the sample to be well dispersed with |
| | nanoparticles clearly visible. The particles exhibited |
| | Brownian motion. Larger acicular crystals were |
| | observed in large numbers, most of which appeared less |
| | than 2000 nm. These exhibited some Brownian motion at |
| | a slower rate than the nanoparticles. These are possibly |
| | resulting from crystal growth or unmilled material. |
| 10 | Microscopy showed the sample to be well dispersed with |
| | nanoparticles exhibiting Brownian motion clearly visible. |
| | A considerable number of larger acicular crystals were |
| | also observed in the sample. |
| 11 | The sample displayed moderate Brownian motion and |
| | was mostly flocculated across the slide. Rod-shaped |
| | particles were visible, with most appearing less than |
| | 2000 nm. |

TABLE 4

| | Mean/ | D50/ | D90/ | D95/ | Mode/ | Median/ | 60 second |
|---|---|---|---|---|---|---|---|
| Sample | nm | nm | nm | nm | nm | nm | sonication |
| 1 | 2328 | 2185 | 4384 | 5285 | 2435 | 2185 | N |
| | 214 | 196 | 308 | 370 | 185 | 196 | Y |
| 2 | 326 | 273 | 452 | 625 | 274 | 273 | N |
| | 259 | 229 | 391 | 486 | 212 | 229 | Y |
| 3 | 195 | 181 | 279 | 326 | 164 | 181 | N |
| | 193 | 180 | 276 | 322 | 164 | 180 | Y |
| 4 | 516 | 307 | 1151 | 1901 | 277 | 307 | N |
| | 259 | 226 | 390 | 491 | 212 | 226 | Y |
| 5 | 2962 | 2804 | 5271 | 6333 | 3176 | 2804 | N |
| | 1073 | 313 | 2570 | 6218 | 277 | 313 | Y |
| 6 | 204 | 189 | 289 | 337 | 184 | 189 | N |
| | 191 | 179 | 269 | 311 | 164 | 179 | Y |
| 7 | 223 | 205 | 320 | 379 | 208 | 205 | N |
| | 224 | 206 | 321 | 379 | 208 | 206 | Y |
| 8 | 321 | 299 | 462 | 539 | 280 | 299 | N |
| | 320 | 299 | 459 | 533 | 281 | 299 | Y |
| 9 | 1057 | 366 | 3222 | 4356 | 314 | 366 | N |
| | 395 | 324 | 587 | 811 | 316 | 324 | Y |
| 10 | 899 | 359 | 2601 | 3443 | 280 | 359 | N |
| | 356 | 313 | 536 | 668 | 314 | 313 | Y |
| 11 | 531 | 306 | 1153 | 2096 | 278 | 306 | N |
| | 295 | 275 | 420 | 493 | 275 | 275 | Y |

TABLE 5

| | Successful formulation | |
|---|---|---|
| Sample | Yes or No | Comments |
| 1 | N (based on PS results | Particle size analysis and microscopy |
| | and microscopy) | were performed on harvested material |
| | | after the 60 min milling processing. |
| 2 | Y (based on PS results | Particle size analysis and microscopy |
| | and microscopy) | were performed on harvested material |
| | | after the 60 min. milling processing. |
| 3 | Y (based on PS results | Particle size analysis and microscopy |
| | and microscopy) | were performed on harvested material |
| | | after the 60 min. milling processing. |
| 4 | Y (based on PS results | Particle size analysis and microscopy |
| | and microscopy). | were performed on harvested material |
| | | after the 60 min. milling processing. |
| 5 | N (based on PS results | Particle size analysis and microscopy |
| | and microscopy) | were performed on harvested material |
| | | after the 60 min. milling processing. |
| 6 | Y (based on PS results | Particle size analysis and microscopy |
| | and microscopy) | were performed on harvested material |
| | | after the 60 min. milling processing. |
| 7 | Y (based on PS results | Particle size analysis and microscopy |
| | and microscopy) | were performed on harvested material |
| | | after the 60 min. milling processing. |
| 8 | Y (based on PS results) | Particle size analysis only was |
| | | performed on harvested material after |
| | | the 60 min milling processing. |
| | | Microscopy was not carried out for |
| | | this formulation. |
| 9 | Y (based on PS results | Particle size analysis and microscopy |
| | and microscopy) | were performed on harvested material |
| | | after the 60 min. milling processing. |
| 10 | Y (based on PS results | Particle size analysis and microscopy |
| | and microscopy) | were performed on harvested material |
| | | after the 60 min. milling processing. |
| 11 | Y (based on PS results | Particle size analysis and microscopy |
| | and microscopy) | were performed on harvested material |
| | | after the 60 min. milling processing. |

It will be apparent to those skilled in the art that various modifications and variations can be made in the methods and compositions of the present inventions without departing from the spirit or scope of the invention. Thus, it is intended that the present invention cover the modification and variations of the invention provided they come within the scope of the appended claims and their equivalents.

The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values are provided for embodiments, additional embodiments are described by taking any 2 different values as the endpoints of a range. Such ranges are also within the scope of the described invention.

All references, patents, and/or applications cited in the specification are incorporated by reference in their entireties, including any tables and figures, to the same extent as if each reference had been incorporated by reference in its entirety individually.

What is claimed is:

1. A stable nanoparticulate tadalafil, or a salt or derivative thereof, composition comprising:
(a) particles of tadalafil having an effective average particle size of less than about 2000 nm; and
(b) at least one surface stabilizer.

2. The composition of claim 1, wherein the tadalafil particle is selected from the group consisting of a crystalline phase, an amorphous phase, a semi-crystalline phase, a semi-amorphous phase, and mixtures thereof.

3. The composition of claim 1, wherein the effective average particle size of the tadalafil particles is selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, and less than about 50 nm.

4. The composition of claim 1, wherein the composition is formulated:
(a) for administration selected from the group consisting of oral, pulmonary, intravenous, rectal, ophthalmic, colonic, parenteral, intracisternal, intravaginal, intraperitoneal, ocular, otic, local, buccal, nasal, bioadhesive and topical administration;
(b) into a dosage form selected from the group consisting of liquid dispersions, gels, aerosols, ointments, creams, lyophilized formulations, tablets, capsules;
(c) into a dosage form selected from the group consisting of controlled release formulations, fast melt formulations, delayed release formulations, extended release formulations, pulsatile release formulations, mixed immediate release formulations, controlled release formulations; or
(d) any combination of (a), (b), and (c).

5. The composition of claim 1, wherein the composition is formulated for administration in a form selected from the group consisting of oral tablets, capsules, sachets, solutions, dispersions and mixtures thereof.

6. The composition of claim 1, wherein the composition further comprises one or more pharmaceutically acceptable excipients, carriers, or a combination thereof.

7. The composition of claim 1, wherein:
(a) tadalafil is present in an amount selected from the group consisting of from about 99.5% to about 0.001%, from about 95% to about 0.1%, and from about 90% to about 0.5%, by weight, based on the total combined weight of tadalafil and at least one surface stabilizer, not including other excipients;
(b) the surface stabilizer is present in an amount selected from the group consisting of from about 0.5% to about 99.999% by weight, from about 5.0% to about 99.9% by weight, and from about 10% to about 99.5% by weight, based on the total combined dry weight of tadalafil and at least one surface stabilizer, not including other excipients; or
(c) a combination thereof.

8. The composition of claim 1, further comprising at least one primary surface stabilizer and at least one secondary surface stabilizer.

9. The composition of claim 1, wherein the surface stabilizer is selected from the group consisting of an anionic surface stabilizer, a cationic surface stabilizer, a zwitterionic surface stabilizer, and an ionic surface stabilizer.

10. The composition of claim 1, wherein the surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-phospholipid, PEG-cholesterol, PEG-cholesterol derivative, PEG-vitamin A, PEG-vitamin E, and random copolymers of vinyl acetate and vinyl pyrrolidone, a cationic polymer, a cationic biopolymer, a cationic polysaccharide, a cationic cellulosic, a cationic alginate, a cationic nonpolymeric compound, a cationic phospholipid, cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quartemary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅dimethyl hydroxyethyl ammonium chloride, C₁₂₋₁₅dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™, ALKAQUA™, alkyl pyridinium salts; amines, amine salts, amine oxides, imide azolinium salts, protonated quaternary acrylamides, methylated quaternary polymers, and cationic guar.

11. The composition of claim 1, wherein the composition has one or more characteristics selected from the group consisting of:
(a) improved bioavailability as compared to conventional tadalafil compositions;
(b) a faster rate of absorption as compared to conventional tadalafil compositions;
(c) a faster onset of therapeutic effect as compared to conventional tadalafil compositions;
(d) the pharmacokinetic profile is not significantly affected by the fed or fasted state of a subject ingesting the composition;
(e) the composition does not produce significantly different absorption levels when administered under fed as compared to fasting conditions; and
(f) administration of the composition to a subject in a fasted state is bioequivalent to administration of the composition to a subject in a fed state.

12. The composition of claim 11, wherein “bioequivalency” is established by:
(a) a 90% Confidence Interval of between 0.80 and 1.25 for both Cₘₐₓ and AUC; or
(b) a 90% Confidence Interval of between 0.80 and 1.25 for AUC and a 90% Confidence Interval of between 0.70 to 1.43 for Cₘₐₓ.

13. The composition of claim 1, wherein:
(a) the Tₘₐₓ of the tadalafil, when assayed in the plasma of a mammalian subject following administration, is less than the Tₘₐₓ for a non-nanoparticulate composition of the same tadalafil, administered at the same dosage;
(b) the Cₘₐₓ of the tadalafil, when assayed in the plasma of a mammalian subject following administration, is greater than the Cₘₐₓ for a non-nanoparticulate composition of the same tadalafil, administered at the same dosage;
(c) the AUC of the tadalafil, when assayed in the plasma of a mammalian subject following administration, is greater than the AUC for a non-nanoparticulate composition of the same tadalafil, administered at the same dosage; or
(d) any combination of (a), (b), and (c).

14. The composition of claim 13, wherein:
(a) the Tₘₐₓ is selected from the group consisting of not greater than about 90%, not greater than about 80%, not greater than about 70%, not greater than about 60%, not greater than about 50%, not greater than about 30%, not greater than about 25%, not greater than about 20%, not greater than about 15%, not greater than about 10%, and not greater than about 5% of the Tₘₐₓ exhibited by a non-nanoparticulate composition of the same tadalafil, administered at the same dosage;
(b) the Cₘₐₓ is selected from the group consisting of at least about 50%, at least about 100%, at least about 200%, at least about 300%, at least about 400%, at least about 500%, at least about 600%, at least about 700%, at least about 800%, at least about 900%, at least about 1000%, at least about 1100%, at least about 1200%, at least about 1300%, at least about 1400%, at least about 1500%, at least about 1600%, at least about 1700%, at least about 1800%, or at least about 1900% greater than the Cₘₐₓ exhibited by a non-nanoparticulate composition of the same tadalafil, administered at the same dosage;
(c) the AUC is selected from the group consisting of at least about 25%, at least about 50%, at least about 75%, at least about 100%, at least about 125%, at least about 150%, at least about 175%, at least about 200%, at least about 225%, at least about 250%, at least about 275%, at least about 300%, at least about 350%, at least about 400%, at least about 450%, at least about 500%, at least about 550%, at least about 600%, at least about 750%, at least about 700%, at least about 750%, at least about 800%, at least about 850%, at least about 900%, at least about 950%, at least about 1000%, at least about 1050%, at least about 1100%, at least about 1150%, or at least about 1200% greater than the AUC exhibited by the non-nanoparticulate formulation of the same tadalafil, administered at the same dosage; or
(d) any combination of (a), (b), and (c).

15. The composition of claim 1, additionally comprising one or more active agents useful for the treatment of sexual dysfunction or erectile dysfunction.

16. The composition of claim 15, wherein the one or more active agents is selected from the group consisting of sildenafil, vardenafil, testosterone, bremelanotide and ginseng.

17. A method of preparing nanoparticulate tadalafil, or a salt or derivative thereof, comprising contacting tadalafil particles with at least one surface stabilizer for a time and under conditions sufficient to provide a nanoparticulate tadalafil composition having an effective average particle size of less than about 2000 nm.

18. The method of claim 17, wherein contacting comprises one or more methods selected from the group consisting of milling, homogenization, precipitation, freezing, supercritical particle generation, and template emulsion.

19. A method for treating a human subject comprising administering to the subject a therapeutically effective amount of the composition of claim 1.

20. The method of claim 19, wherein the subject has a disease, condition or symptoms selected from the group consisting of: sexual dysfunction, erectile dysfunction, pulmonary arterial hypertension, myocardial infarction, ischemia/reperfusion injury, chronic obstructive pulmonary disease (COPD), adult respiratory distress syndrome (ARDS), acute lung injury (ALI), bronchitis, bronchial asthma, pulmonary fibroses, emphysema, interstitial pulmonary disorder, pneumonia, or a combination thereof.
